(19) United States
(12) Patent Application Publication
(10) Pub. No.: US 2008/0207916 A1
(43) Pub. Date: Aug. 28, 2008

(54) Radiolabelled Phenylethyl Imidazole Carboxylic Acid Ester Derivatives

(75) Inventors: Ilse Zolle, Wien (AT); Friedrich Hammerschmidt, Wien (AT)

Correspondence Address: LERNER GREENBERG STEMER LLP, P O BOX 2480, HOLLYWOOD, FL, 33022-2480, US

(73) Assignee: Ilse Zolle, Vienna (AT)

(21) Appl. No.: 12/050,627

(22) Filed: Mar. 18, 2008

Related U.S. Application Data
(63) Continuation-in-part of application No. 11/582,073, filed on Oct. 17, 2006, now Pat. No. 7,358,369.
Application No. 11/582,073 is a division of application No. 10/635,294, filed on Aug. 6, 2003, now Pat. No. 7,189,859.

(51) Int. Cl. C07D 233/90; C12P 7/22

(52) U.S. Cl. 548/334.5; 435/156

(57) ABSTRACT

The invention relates to radioactively labeled derivatives of (R)-1-(1-phenylethyl)-1H-imidazole-5-carboxylic acid esters, uses, and methods for preparing these compounds.

### CROSS-REFERENCE TO RELATED APPLICATIONS

This is a continuation-in-part application of copending patent application Ser. No. 11/582,073, filed Oct. 17, 2006, which is a divisional of patent application Ser. No. 10/635,294, filed Aug. 6, 2003, now U.S. Pat. No. 7,189,859 B2; the prior applications are incorporated herein by reference in their entirety.

### BACKGROUND OF THE INVENTION

### Field of the Invention

### Synthesis of Compounds for Adrenal Scintigraphy

### BRIEF SUMMARY OF THE INVENTION

The invention relates to previously disclosed radioactively labeled derivatives of (R)-1-(1-phenylethyl)-1H-imidazole-5-carboxylic acid esters and methods for preparing these compounds. The invention also relates to the use of these radioactively labeled compounds as radiopharmaceuticals for functional diagnosis of adrenal disease and for therapeutic applications. In particular, these compounds bind selectively to adrenocortical tissue facilitating the diagnosis of adrenal cortical masses such as incidentaloma, adenoma, primary and metastatic cortical carcinoma.

With the foregoing and other objects in view there is provided, in accordance with the invention, a process for preparing the compounds represented by formula (IB, see below) comprising the steps:
(A) preparing an (S)-secondary alcohol of formula (III)

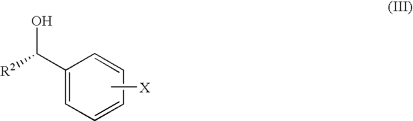

(B) coupling said (S)-secondary alcohol of formula (III) obtained in step (A) with an alkyl imidazole-5-carboxylate of formula (IV)

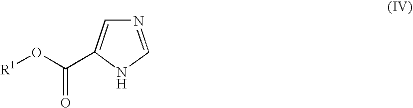

wherein
R¹ represents a straight or branched alkyl chain containing from 1 to 4 carbon atoms, wherein the alkyl group is optionally substituted with a halogen;
R² represents a straight alkyl chain in (S)-configuration containing from 1 to 2 carbon atoms;
X is a halogen.
   The reaction proceeds with inversion of configuration, producing the halogenated compound of formula (IB);

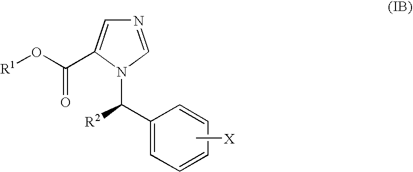

wherein
R¹ is linear or branched C₁-C₄ alkyl, and is optionally substituted with a halogen, selected from the groups consisting of F, Cl, I or Br;
R² denotes an alkyl group in (R)-configuration containing from 1 to 2 carbon atoms; and
X is a halogen, selected from the groups consisting of I, Br, F, or Cl; preferably iodine.

In accordance with another feature of the invention, there is provided a process for preparing the intermediary (S)-1-(4-iodophenyl)ethanol of formula (III) by lipase SAM II-catalyzed resolution, comprising the steps:
a. reducing a substituted phenyl alkyl ketone to the corresponding racemic alcohol;
b. preparing the chloroacetate of said racemic alcohol; and
c. performing a lipase SAM II-catalyzed resolution of (S)-III derived from
d. the (S)-enantiomeric ester;
   The synthesis of (S)-III is outlined in scheme 1.

In accordance with yet another feature of the invention there is provided a process for preparing the compounds labelled in the phenyl ring, the method comprising:
(a) preparing a stannylated precursor of formula (II);
(b) subjecting said compound to a standard procedure for replacing the alkylstannyl group (L) with a radiohalogen, producing a compound of the general formula (IC):

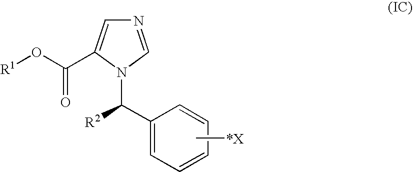

wherein
R¹ is linear or branched C₁-C₄ alkyl, optionally substituted with a halogen selected from F, Cl, I or Br;
R² denotes an alkyl group containing 1 or 2 carbon atoms; and
*X is a radioactive halogen, selected from the group consisting of ¹²³I, ¹²⁴I, ¹²⁵I ¹³¹I, ⁷⁶Br, ⁸²Br, ²¹¹At, or ¹⁸F.

In accordance with an added feature of the invention, there is provided a process for preparing a stannylated precursor of the general formula (II) by reacting the compound obtained in claim 4, step (B) with a stannylation agent in the presence of a catalyst, said stannylated precursor being represented by the following formula:

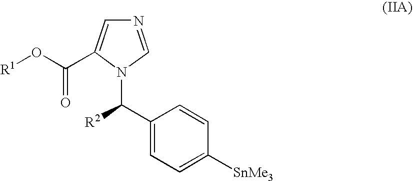

wherein
R¹ and R² are as defined above;
L represents a trimethylstannyl substituent.

### DETAILED DESCRIPTION OF THE INVENTION

The present invention provides a compound of the general formula I:

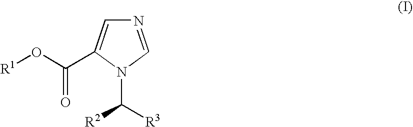

wherein
R¹ is linear or branched C₁-C₄ alkyl, and is optionally substituted with a halogen selected from the groups consisting of F, Cl, I or Br;
R² denotes an alkyl group containing 1 or 2 carbon atoms; and
R³ is phenyl, optionally substituted with a halogen;

As used herein, the expression “alkyl” includes methyl and ethyl groups, and linear or branched propyl groups. Particular alkyl groups are methyl, ethyl, 2-fluoroethyl, n-propyl, and 2-propyl.

The term “halogen” as used herein, includes iodine, bromine, chlorine, fluorine, and astatine.

The substituent R¹ on the carboxylic ester group may be transformed to other substituents encompassed by the definition of R¹ by suitable reactions known in the art for the modification of carboxylic acid functions, i.e., by hydrolysis and esterification and/or transesterification. The starting materials for the preparation of the novel compounds of formula (I) are known or they have been obtained by enantioselective synthesis disclosed herein.

Particularly preferred novel compounds in accordance with the present invention are those ester compounds wherein R¹ is alkyl substituted with a halogen, preferably with a radioactive halogen.

The compound of formula (I) in accordance with the present invention is suitably a radiolabeled derivative of formula IA:

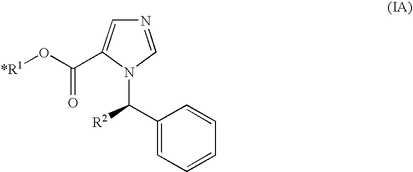

wherein
R¹ is linear or branched C₁-C₄ alkyl, and is optionally substituted with an □-halogen; said halogen being selected from fluorine, preferably radioactive fluorine;
R² denotes an alkyl group containing 1 or 2 carbon atoms; and
R³ is phenyl;

Preferred are compounds of formula IA, wherein R¹ is 2-fluoroethyl and said halogen is ¹⁸F.

The compound of formula (I) in accordance with the present invention is suitably a compound wherein phenyl is substituted with a halogen of formula IB:

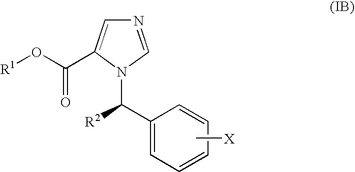

wherein
R¹ is linear or branched C₁-C₄ alkyl, and is optionally substituted with a halogen, selected from the groups consisting of F, Cl, I or Br;
R² denotes an alkyl group containing 1 or 2 carbon atoms; and
X is a halogen, selected from the groups consisting of I, Br, F, or Cl;

Halogenated compounds of formula IB serve as intermediary derivatives for the synthesis of stannylated precursors, which provide access to radiolabelled derivatives of formula IC:

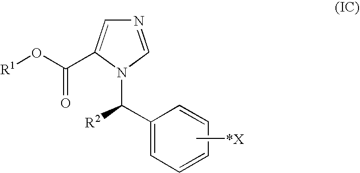

wherein
R¹ is linear or branched C₁-C₄ alkyl, and is optionally substituted with a halogen selected from the groups consisting of F, Cl, I or Br;
R² denotes an alkyl group containing 1 or 2 carbon atoms; and
X is a radioactive halogen, selected from the group consisting of, ¹²³I, ¹²⁴I, ¹²⁵I ¹³¹I, ⁷⁶Br, ⁸²Br, ²¹¹At, or ¹⁸F;

Preferred are compounds of formula IC, wherein said halogen is ¹²³I, ¹²⁴I ¹³¹I, or ¹⁸F.

The present invention includes within its scope stannylated derivatives of formula II:

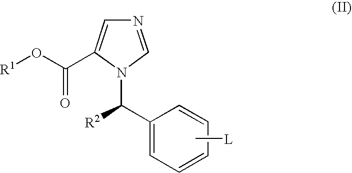

wherein
R¹ is linear or branched C₁-C₄ alkyl, optionally substituted with a halogen selected from F, Cl, I or Br;
R² denotes an alkyl group containing 1 or 2 carbon atoms; and
L represents trimethylstannyl, triethylstannyl, tri-n-propylstannyl, and tri-n-butylstannyl;

Compounds of formula II contain a leaving group L, suitably selected from trimethylstannyl, triethylstannyl, tri-n-propylstannyl, and tri-n-butylstannyl, especially trimethylstannyl. Stannylated derivatives are key intermediates for radiotracer synthesis by oxidative radiohalogenido destannylation. The trialkylstannyl group in an aromatic ring is replaced under mild conditions by radiohalogen to yield the respective radioactively labelled compounds of formula (IC).

The compound of formula (II) in accordance with the present invention is suitably a compound wherein L is the trimethylstannyl substituent of formula IIA:

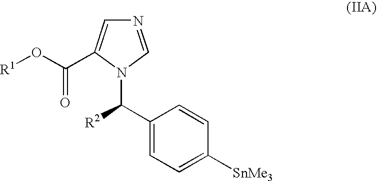

wherein R¹ and R² are as defined above.

The invention concerns a process of synthesizing compounds of formula (I) by a stereoselective and regioselective new approach.

The compounds according to the invention may be prepared by a process, which comprises coupling a compound of formula III:

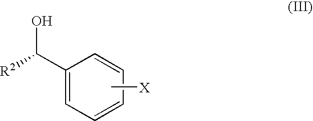

with a compound of formula IV:

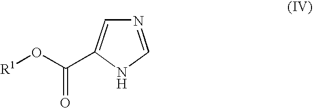

wherein
R¹ represents a straight or branched alkyl chain containing from 1 to 4 carbon atoms, wherein the alkyl group is optionally substituted with a halogen;
R² represents a straight alkyl chain in (S)-configuration containing from 1 to 2 carbon atoms;
X is a halogen.

The reaction between compounds III and IV proceeds with inversion of configuration, to give compounds of formula IB:

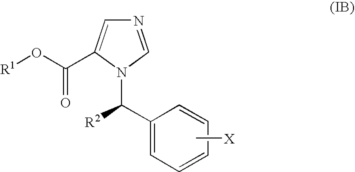

wherein
R¹ is linear or branched C₁-C₄ alkyl, and is optionally substituted with a halogen, selected from the groups consisting of F, Cl, I or Br;
R² denotes an alkyl group containing 1 or 2 carbon atoms; and
X is a halogen, selected from the groups consisting of I, Br, F, or Cl;

The reaction between compounds III and IV is based on the known Mitsunobu reaction (Mitsunobu, 1981). The alcohol (S)-1-(4-iodophenyl)ethanol (III) is reacted with methyl imidazole-5-carboxylate (IV) in the presence of triphenylphosphane and a dialkyl azodicarboxylate (preferably di-t-butyl azodicarboxylate). Triphenyl phosphinoxide and the hydrazo ester are by-products of the reaction. The reaction conditions favour activation of the alcohol to generate the reactive alkoxyphosphonium salt. Methyl imidazole-5-carboxylate is expected to be deprotonated, thus N−1 and N−3 could react as nucleophile with the alkoxyphosphonium cation to give a mixture of two isomeric N-substituted imidazoles. Yet, when coupling III and IV at low temperature, alkylation is observed exclusively at N-3 with clean inversion.

(S)-III is a key intermediate and needs to be synthesized; methyl imidazole-5-carboxylate IV is commercially available.

Since not commercially available, the (S)-alcohol of formula III is prepared by a novel synthetic approach, described hereafter and in scheme 1: Starting from 4-iodophenyl methyl ketone, which is reduced to the racemic alcohol and converted to (±)-1-(4-iodophenyl)ethyl chloroacetate, the racemic ester is subjected to stereoselective enzyme hydrolysis. The remaining (S)-isomer of the ester is separated from the (R)-alcohol and transesterified to give (S)-1-(4-iodophenyl)ethanol (ee >98%) III.

Laumen & Schneider (1988) reported that lipase SAM II hydrolizes acetates and chloroacetates of secondary benzyl alcohols with high enantioselectivity, therefore, Schneider's procedure is applied to the resolution of racemic 1-(4-iodophenyl)ethanol. Lipase SAM II is known to hydrolyze preferentially the (R)-esters of secondary benzylic alcohols; however, application with the substrate described in the invention is new.

(R)-4-Iodo-metomidate is derived exclusively from the (S)-alcohol of the ester, which is not accepted as substrate by lipase SAM II. Coupling of (S)-1-(4-iodophenyl)ethanol with methyl imidazole-5-carboxylate yields (R)-4-iodo-metomidate with clean inversion.

This novel reaction offers a versatile approach to the synthesis of compounds described by formula I. (S)-1-(4-iodophenyl)ethanol (ee >98%) III is synthesized by lipase-catalyzed resolution and coupled with methyl imidazole-5-carboxylate IV. The two fragments are joined regioselectively at N−3 with clean inversion of configuration producing (R)-methyl 3-[1-(4-iodophenyl)ethyl]-1H-imidazole-5-carboxylate (4-iodo-MTO).

Compounds of formula IA and IB, labeled with a radiohalogen are obtained by alternate routes. Compounds of formula IA typically represent radiohalogenated esters, whilst compounds of formula IB comprise the various esters of phenyl halogenated derivatives.

The substituent R¹ on the carboxylic ester group may be transformed to other substituents defined as R¹ by suitable reactions known in the art for the modification of carboxylic acid functions. However, introducing a positron emitter requires special techniques, esterification is generally performed on-line using closed synthesis modules.

Compounds containing a radiolabelled phenyl ring R³ are labelled by oxidative destannylation of especially synthesized precursors, which facilitate rapid labelling under mild reaction conditions. Therefore, 4-iodo-metomidate or 4-iodo-etomidate, respectively, is converted to the 4-trimethylstannyl derivative of formula IIA to serve as a precursor for labelling metomidate and etomidate with a suitable radiohalogen.

Compounds of formula II wherein L represents a leaving group, may be prepared by standard stannylation techniques. The exchange of halogen for the trialkylstannyl substituent (L) is catalyzed by tetrakis (triphenylphosphane) palladium to yield a stannylated compound of formula IIA.

Radiohalogenated compounds of formula IC are conveniently prepared with high specific activities by reacting a compound of formula IIA with a radiohalogen (Iodine-123; iodine-131; bromine-76, fluorine-18, and others) in the presence of an oxidizing agent, at room temperature.

The radioligand ¹³¹I-MTO is produced with a specific activity of >50 GBq/μmol, resp. >1.35 Ci/μmol.

The compounds in accordance with the present invention potently and selectively bind to adrenocortical membranes (cytochrome P-450c11).

The present invention is described below in more detail in connection with the synthesis of an R¹ derived labelled ester, namely (R)-2-[¹⁸F]fluoroethyl 1-(1-phenylethyl)-1H-imidazole-5-carboxylate and R³ derived radiotracer (R)-1-[1-(4-[¹³¹I]iodophenyl)ethyl]-1H-imidazole-5-carboxylic acid methyl ester (¹³¹I-IMTO); The example is given merely for illustrative purposes and shall in no way be understood as a limitation of the scope of the present invention which is given by the patent claims.

### EXAMPLES

### Preparation of (R)-1-[1-(4-¹³¹I-iodophenyl)ethyl]-1H-imidazole-5-carboxylic acid methyl ester (¹³¹I-IMTO)

### a) Synthesis of (S)-1-(4-iodophenyl)ethanol (III)

The substituted (S)-alcohol was prepared according to scheme 1:

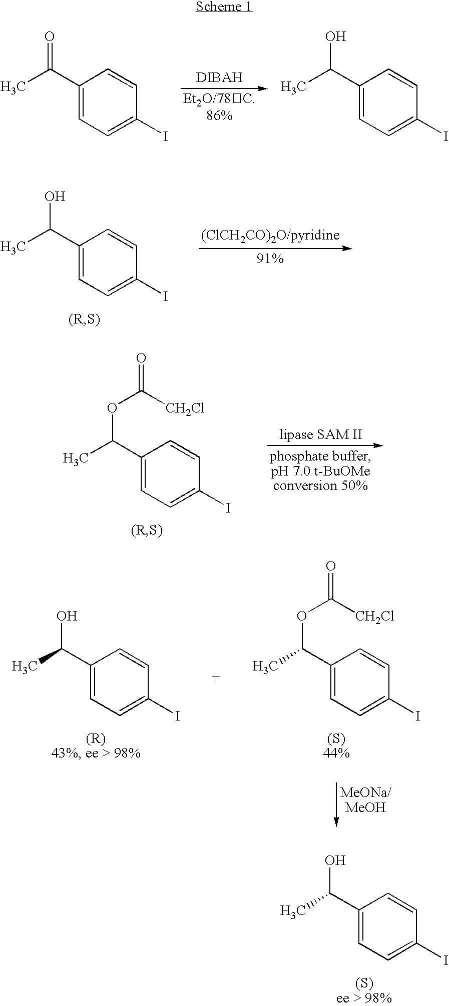

### b. Mitsunobu Reaction of benzylic alcohols with methyl 1H-imidazole-5-carboxylate

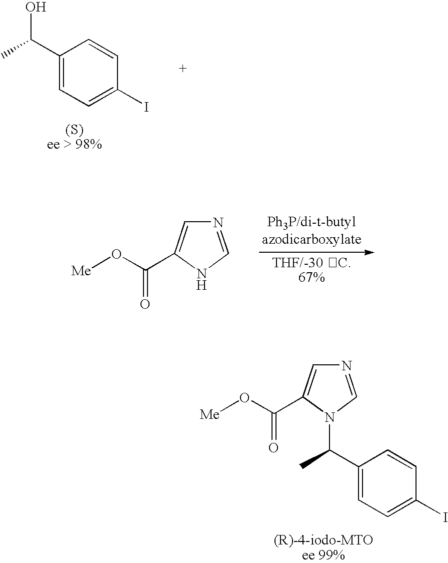

### c. Synthesis of (R)-(+)-Methyl 1-[1-(4-iodophenyl)ethyl]-1H-imidazole-5-carboxylate

A solution of substituted (S)-alcohol (1.98 g, 7.98 mmol, ee>98%) in dry THF (14.5 cm³) was added dropwise to a stirred solution of methyl 1H-imidazole-5-carboxylate (1.008 g, 7.98 mmol) and triphenylphosphane (2.503 g, 9.43 mmol) in dry THF (22.0 cm³) in an atmosphere of argon −30° C. Then, a solution of di-t-butyl azodicarboxylate (2.204 g, 9.57 mmol) in dry THF (14.5 cm³) were added and the stirred reaction mixture was allowed to warm up from −30° C. to 0° C. within 2.5 hr. No alcohol could by detected by TLC (diethyl ether-diisopropylamine 10:1). The reaction mixture was concentrated under reduced pressure. The residue was mixed with diethyl ether (36 cm³) and stirred for 2 h. The crystals (triphenylphosphanoxide and hydrazo ester) were collected and washed with diethyl ether (3×15 cm³). The filtrate was evaporated and reduced pressure to leave a residue, which was purified by flash chromatography (hexanes-diethyl ether-diisopropylamine 50:30:1; TLC: diethyl ether-diisopropylamine 10:1, R_{f}=0.44 for iodide, 0.54 for metomidate) on silica gel to give p-iodo-metomidate (1.91 g, 67%, ee 99%); [a]²⁰D=+76.0 (c 1.09 in acetone). Anal. (C₁₃H₁₃IN₂O₂) C, H, N.

### Radiosynthesis of 4-[¹²³1]iodophenyl-metomidate

Generally, the synthesis of radiolabeled phenyl derivatives is performed shortly before use by oxidative radiohalogenido destannylation of a suitable precursor molecule. Substitution with a radiohalogen in the phenyl ring offers access to diagnostic as well as therapeutic MTO-derivatives. Radionuclides for therapy are beta- and alpha-emitting halogens, e.g., ¹³¹I, ⁸²Br, and ²¹¹At.

### d. Synthesis of precursor (R)-1-[1-(4-trimetylstannylphenyl)ethyl]-1H-imidazole-5-carboxylic acid methyl ester (IIA)

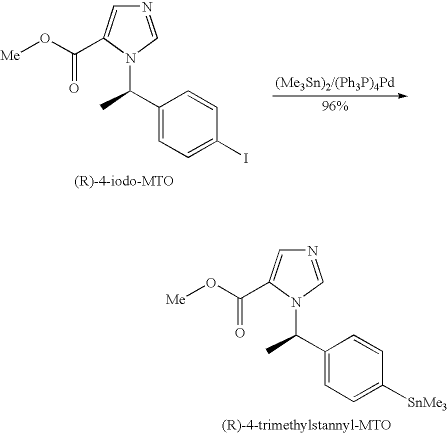

Hexamethylditin (0.645 g. 3.2 mmol, 6.5 cm³ of a solution of 1.0 g hexamethylditin in 10 cm³ of dry toluene), tetrakis(triphenylphosphane)palladium (58 mg, 5 mol %) and triethylamine (1.6 cm³, 11.6 mmol) were added to a stirred solution of iodometomidate (0.368 g. 1.03 mmol, ee >98%) in an atmosphere of argon and refluxed (bath temperature 135° C.) for 17 hr. The cooled solution was concentrated under reduced pressure and the residue was purified by flash chromatography (hexane-diethyl ether-diisopropylamine 60:30:1; TLC:diethyl ether-diisopropylamine 10:1, R_{f}=0.71 for stannane, R_{f}=0.50 for iodometomidate) on silica gel to give stannane (0.377 g, 96%) as a crystalline solid (Found: C, 48.7; H, 5.6 N, 7.1, C₁₆H₂₂N₂O₂Sn requires C, 48.9; H, 5.6; N, 7.1%); mp 77-79° C. (from hexane); [□]²⁰D=+82.09 (c 2.06 in acetone). νₘₐₓ _{(Si, film)/cm}⁻¹ 2981, 1715, 1437, 1362, 1218, 1133, 1110, 1049; δ_{H} (400.13 MHz, CDCl₃) 0.25 (9H, s, (CH₃)₃Sn, ^{117/119}Sn satellites, 2×d, J 53.2 and 55.2), 1.82 (3H, d, J 7.0, CH₃CH), 3.77 (3H, s, OCH₃), 6.30 (1H, q, J 7.0, CH₃CH), 7.13 (2H, d, J 8.0, 2×Hₐᵣₒₘ, ^{117/119}Sn satellites, d, J 9.0), 7.43 (2H, d, J 8.0, 2×Hₐᵣₒₘ, ^{117/119}Sn satellites, d, J 42.7), 7.71 (1H, s, Hₕₑₜₐᵣₒₘ), 7.74 (1H s, Hₕₑₜₐᵣₒₘ); δ^{C} (100.61 MHz, CDCl₃) -9.61 (3 C, (CH₃)₃Sn), 22.16 (CH₃CH), 51.40 (OCH₃), 55.30 (CH₃CH), 122.31 (Cₐᵣₒₘ), 125.81 (2×C, 2×HCₐᵣₒₘ), ^{117/119}Sn satellites, d, J 45.9), 136.28 (2×C, 2×HCₐᵣₒₘ, ^{117/119}Sn satellites, d, J 36.8), 122.29 (2 C, 2×HCₐᵣₒₘ), 128.02 (2 C, 2×HCₐᵣₒₘ), 137.94 (Cₐᵣₒₘ), 138.23 (HCₕₑₜₐᵣₒₘ), 139.84 (HCₕₑₜₐᵣₒₘ), 141.00 (Cₐᵣₒₘ or CCO), 142.24 (CCO or Cₐᵣₒₘ), 160.68 (CO).

Compounds of the general formula (IB) are obtained by Mitsunobu Coupling (Scheme 1). (R)-iodometomidate is converted by treatment with hexaalkyldistannanes and a palladium catalyst to yield the stannylated precursor of formula (IIA).

e. Synthesis of Modified Esters
Transesterification of commercially available etomidate at ambient temperature in dry MeOH, n-propanol, or 2-propanol in the presence of the corresponding sodium alkoxide yielded metomidate, and the n-propyl and 2-propyl esters, respectively.

### f. Synthesis of (R)-2-fluoroethyl 1-(1-phenylethyl)-1H-imidazole-5-carboxylate

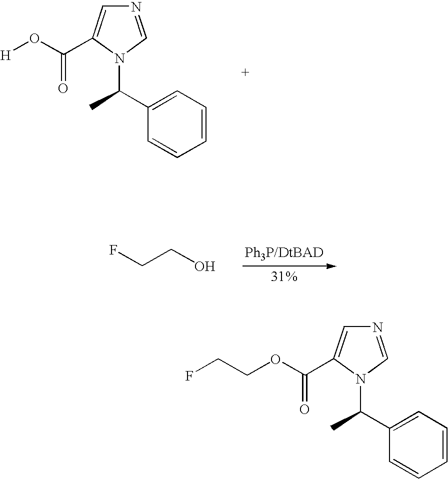

A solution of DtBAD (0.128 g, 0.554 mmol) in dry toluene (2 mL) was added to a stirred mixture of Ph₃P (0.145 g, 0.554 mmol), methyl 1H-imidazole-5-carboxylate (0.100 g, 0.462 mmol) and 2-fluoroethanol (44 mg, 0.040 mL, 0.681 mmol) dry toluene (2 mL) under an atmosphere of argon. After 18 h, water (two drops) was added and the mixture was concentrated under reduced pressure to give a residue which was purified by flash chromatography (first column: 60 g of silica gel, hexane/Et₂O/iPr₂NH 5/10/1, R_{f} 0.25, 98 mg of mixture of 2-fluoroethyl ester and hydrazo ester; second flash chromatography: 40 g silica gel, Et₂O as eluent, R_{f} 0.30) to give the product (38 mg, 31%) as a crystalline solid, mp 51° C. (hexane); [□]²⁰_{D}=+106.29 (c 0.72, acetone). Anal. (C₁₄H₁₅FN₂O₂) C, H, N.

### Radiosynthesis of (R)-2-[¹⁸F]fluoroethyl 1-(1-phenylethyl)-1H-imidazole-5-carboxylate

Synthesis is based on the nucleophilic radiofluorination with no-carrier-added ¹⁸F-fluoride after kryptofix 2.2.2.-activated nucleophilic substitution of 1,2-dibromoethane in acetonitrile to yield 2-[¹⁸F]fluoroethyl bromide for ¹⁸F-fluoroethylation of (R)-1-(1-phenylethyl)-1H-imidazole-5-carboxylic acid as the tetrabutylammonium salt to yield the labeled fluoroethyl derivative (¹⁸F-FETO). The radioligand is produced with a specific activity of approx. 40 GBq/μmol (1.1 Ci/μmol).

¹⁸F-fluoroethylation requires special techniques for the conversion of the radionuclide to reactive 2-¹⁸F-fluoroethyl bromide for subsequent esterification, which is generally performed at the site of radionuclide production.

While the invention has been described in its preferred form or embodiment with some degree of particularity, it is understood that this description has been given only by way of example and that numerous changes in the details of synthesis, fabrication, and use, including the combination and arrangement of parts, may be made without departing from the spirit and scope of the invention.

### LITERATURE RELATED TO THE PRIOR ART

Basmadjian G P, Hetzel K R, Ice R D, Beierwaltes W H (1975) Synthesis of a new adrenal cortex imaging agent 6□-[¹³¹I]-iodomethyl-19-norcholest-5(10)en-3□-ol (NP-59). J. Labelled Compd. & Radiopharm. XI: 427-434.
Sakar S D, Ice R D, Beierwaltes W H, Gill S P, Balanchandran S, Basmadjian G P (1976) Selenium-75-19-selenocholesterol—a new adrenal scanning agent with high concentration in the adrenal cortex. J Nucl Med 17: 212-217.
Beierwaltes, W. H., Wieland, D. M., Ice, R. D., Seabold, J. E., Sarkar, S. D., Gill, S. P., Mosley, S. T. (1976) Localization of radiolabeled enzyme inhibitors in the adrenal gland. J. Nucl. Med., 17(11), 998-1002.
Beierwaltes, W.H., Wieland, D. M., Mosley, S.T., Swanson, D. P., Sarkar, S. D., Freitas, J. E., Thrall, J. H, Herwig, K. R. (1978) Imaging the adrenal glands with radiolabeled inhibitors of enzymes: concise communication. J. Nucl. Med. 19(2), 200-203.
Wu J. L., Wieland D. M., Beierwaltes W. H., Swanson D. P., Brown L. E.: Radiolabelled enzyme inhibitors—enhanced localization following enantiomeric purification. J. Labelled Compd. & Radiopharm., XVI(1), 6-9,1979.
Wieland D M: Radiolabeled enzyme inhibitors—Adrenocortical enzymes. In: Receptor-binding radiotracers, Vol. I, 127-146, Ed. W. C. Eckelman, Chemical Rubber Co. Press, Cleveland, Ohio, 1982.
Robien W. and Zolle I. (1983) Synthesis of radioiodinated metyrapone—A potential agent for functional imaging of the adrenal cortex. Int. J. Appl. Radiat. Isot. 34: 907-914.
Allolio B, Stuttmann R, Fischer H, Leonhard W, Winkelman W (1983) Long-term etomidate and adrenocortical suppression. The Lancet ii, 626.
Zolle, I., Woloszczuk, W., Höfer, R. Synthesis and in vitro evaluation of metyrapone derivatives as potential inhibitors of 11□-hydroxylase activity. In: Radiopharmaceuticals and labelled compounds, 337-342, IAEA-CN-45/67, Vienna, 1985.
Yu, J., Zolle, I., Mertens, J., Rakias, F.: Synthesis of 2-¹³¹I-iodophenyl-metyrapone using Cu(I)-assisted nucleophilic exchange labelling: Study of the reaction conditions. Nucl. Med. & Biol. 22(2): 257-262 (1995).
Vanden Bossche H, Willemsens G, Cools W, Bellens D (1984) Effects of etomidate on steroid biosynthesis in subcellular fractions of bovine adrenals. Biochemical Pharmacology: 33(23), 3861-3868.
Gross M D, Shapiro B. The adrenal cortex In: Principles of Nuclear Medicine, 2^{nd} ed. H. N. Wagner, Jr., S. Szabo, J. W. Buchanan, eds. Philadelphia: W B Saunders, pp. 652-664, 1995.
Belelli, D.; Lambert, J. J.; Peters, J. A.; Wafford, K.; Whiting, P. J. (1997) The interaction of the general anesthetic etomidate with the g-aminobutyric acid type A receptor is influenced by a single amino acid. Proc. Natl. Acad. Sci. USA 94, 11031-11036.
Franks, N. P. (2006) Molecular targets underlying general anaesthesia. Br. J. Pharmacol. 147 Suppl 1, S72-81.
Engelhardt D (1994) Steroid biosynthesis inhibitors in Cushing's syndrome. Clin Investig 72: 481-488.
Weber M M, Lang J, Abedinpour F, Zeilberger K, Adelmann B, Engelhardt D (1993) Different inhibitory effect of etomidate and ketoconazole on the human adrenal steroid biosynthesis. Clin. Invest. 71: 933-938.
Godefroi, E. F., Janssen, P. A. J., Van der Eycken, C. A. M., Van Heertum, A. H. M. T., Niemegeers, C. J. E. (1965) DL-1-(1-Arylalkyl)imidazole-5-carboxylate esters. A novel type of hypnotic agents. J. Med. Chem. 8: 220-223.
Synthesis of etomidate U.S. Pat. No. 3,354,173 issued Nov. 21, 1967. Expired Nov. 1984.

### Reviews:

Mitsunobu O. (1981) Synthesis: 1-28.
Hughes D. L. (1992) Org. Reactions 42: 335-656.
Hughes D. L. (1996) Org. Prep. Proced. Int. 28: 127-164.

### Reference for Iodinations:

Merkushev E. B. (1988) Synthesis: 923-937.

### Reference for Radioiodinations:

Ali H. and van Lier J. E. (1996) Synthesis: 423-445.

### Ref. for Destannylation

Baldwin R. M., Zea-Ponce Y., Zoghbi S. S., Laruelle M., Al-Tikriti M. S., Sybirska E. H., Malison R. T., Neumeyer J. L., Milius R. A., Wang S., Stabin M., Smith E. O., Charney D. S., Hoffer P. B., and Innis R. B., (1993) Evaluation of the monoamine uptake site ligand [¹²³I]methyl-3□-(4-iodophenyl)-tropane-2□-carboxylate ([¹²³I]□-CIT) in non-human primates: pharmacokinetics, biodistribution and SPECT brain imaging coregistered with MRI. Nucl. Med. Biol. 20: 597-606.

### Literature for Enzymatic Hydrolysis:

Laumen K. and Schneider M. P. (1988) J. Chem. Soc. Chem. Comm.: 598-800.
   References for Coupling with Mitsunobu:
Botta M., Summa V., Trapassi G., Monteagudo E., Corelli F. (1994) Tetrahedron: Asymmetry 5: 181-184.
Corelli F., Summa V., Brogi A., Monteagudo E., Botta M. (1995) J. Org. Chem. 60: 2008-2015.

### Lit. for Fluoroethylation

Wester H J, Herz M, Weber W, Heiss P, Senekowitsch-Schmidtke R, Schwaiger M, Stocklin G (1999) Synthesis and radiopharmacology of O-(2-¹⁸F-fluoroethyl)-L-tyrosine for tumor imaging. J. Nucl. Med. 40: 205-212.
Wester H-J, Willoch F, Tölle T R, Munz F, Herz M, Øye I, Schadrack J, Schwaiger M, Bartenstein P (2000) 6-O-(2-[¹⁸F]fluoroethyl)-6-O-desmethyldiprenorphine (¹⁸F]DPN): Synthesis, biologic evaluation, and comparison with [¹¹C]DPN in humans. J Nucl Med 41: 1279-1286.
Hamacher, K., Coenen, H. H. (2002) Efficient routine production of the ¹⁸F-labelled amino acid O-(2-¹⁸F-fluoroethyl)-L-tyrosine. Appl. Radiat. Isot. 57: 853-856.
Wadsak, W., Mitterhauser, M., Zolle, I. (2002) Synthesis of [¹⁸F]FETO, a novel PET-tracer for adrenal scintigraphy. Eur. J. Nucl. Med. & Molec. Imaging Vol. 29: Suppl. 1, S59 (Abstract).
Wadsak W, Mitterhauser M (2003) Synthesis of [¹⁸F]FETO, a novel potential 11□-hydroxylase inhibitor. J. Lab. Compd. Radiopharm. 46: 379-388.

1. A compound of the general formula (I) 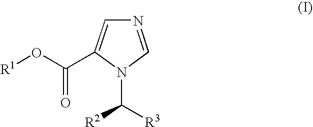
wherein
R¹ is linear or branched C₁-C₄ alkyl, and is optionally substituted with a halogen selected from the groups consisting of F, Cl, I or Br;
R² denotes an alkyl group containing 1 or 2 carbon atoms; and
R³ is phenyl, optionally substituted with halogen.

2. The compound of formula (I), wherein the ester is substituted with a halogen, suitably as a radiolabelled ester derivative of formula IA: 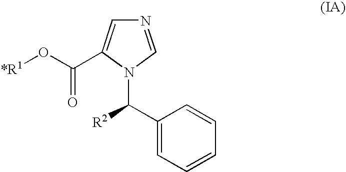
wherein
R¹ is linear or branched C₁-C₄ alkyl, and is optionally substituted with an □-halogen; said halogen being radioactive;
R² denotes an alkyl group containing 1 or 2 carbon atoms.

3. The compound of claim 2, wherein R¹ is radioactive 2-fluoroethyl, R² is methyl and R³ is phenyl; wherein the compound is ¹⁸F-etomidate.

4. A process for preparing the compounds represented by formula (IB) comprising the steps:
(A) preparing an (S)-secondary alcohol of formula (III) 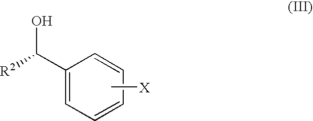
(B) coupling said (S)-secondary alcohol of formula (III) obtained in step (A) with an alkyl imidazole-5-carboxylate of formula (IV) 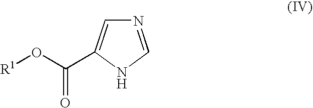
wherein
R¹ represents a straight or branched alkyl chain containing from 1 to 4 carbon atoms, wherein the alkyl group is optionally substituted with a halogen;
R² represents a straight alkyl chain in (S)-configuration containing from 1 to 2 carbon atoms;
X is a halogen.
The reaction proceeds with inversion of configuration, producing the halogenated compound of formula (IB); 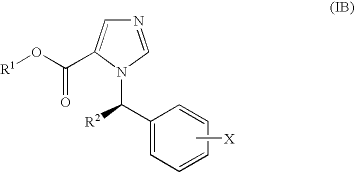
wherein
R¹ is linear or branched C₁-C₄ alkyl, and is optionally substituted with a halogen, selected from the groups consisting of F, Cl, I or Br;
R² denotes an alkyl group in (R)-configuration containing from 1 to 2 carbon atoms; and
X is a halogen, selected from the groups consisting of I, Br, F, or Cl; preferably iodine.

5. A process for preparing the intermediary (S)-1-(4-iodophenyl)ethanol of formula (III) by lipase SAM II-catalyzed resolution, comprising the steps:
(a) reducing a substituted phenyl alkyl ketone to the corresponding racemic alcohol;
(b) preparing the chloroacetate of said racemic alcohol; and
(c) performing a lipase SAM II-catalyzed resolution of (S)-III derived from the (S)-enantiomeric ester;
The synthesis of (S)-III is outlined in scheme 1.

6. A process for preparing the compounds labelled in the phenyl ring, the method comprising:
(a) preparing a stannylated precursor of formula (II);
(b) subjecting said compound to a standard procedure for replacing the alkylstannyl group (L) with a radiohalogen, producing a compound of the general formula (IC): 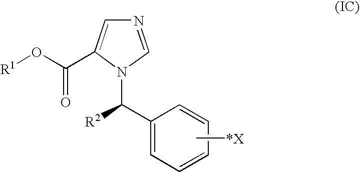
wherein
R¹ is linear or branched C₁-C₄ alkyl, optionally substituted with a halogen selected from F, Cl, I or Br;
R² denotes an alkyl group containing 1 or 2 carbon atoms; and
*X is a radioactive halogen, selected from the group consisting of ¹²³I, ¹²⁴I, ¹²⁵I ¹³¹I, ⁷⁶Br, ⁸²Br, ²¹¹At, or ¹⁸F.

7. A compound of the general formula (II) 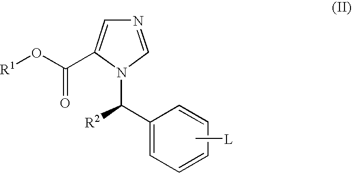
wherein
R¹ is linear or branched C₁-C₄ alkyl, optionally substituted with a halogen selected from F, Cl, I or Br;
R² denotes an alkyl group containing 1 or 2 carbon atoms; and
L represents an alkyl stannyl group selected from the group consisting of trimethylstannyl, triethylstannyl, tri-n-propylstannyl, and tri-n-butylstannyl.

8. A process for preparing a stannylated precursor of the general formula (II) by reacting the compound obtained in claim 4, step (B) with a stannylation agent in the presence of a catalyst, said stannylated precursor being represented by formula (IIA); 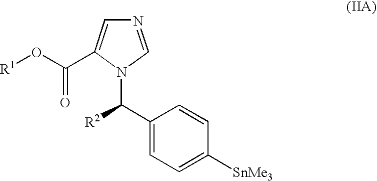
wherein
R¹ and R² are as defined above;
L represents a trimethylstannyl substituent.

9. A compound of claim 7 with the general formula (IIA).

10. A stannylated precursor of claim 7, wherein R¹ and R² are each methyl and L is a trimethylstannyl group.

11. A stannylated precursor of claim 7, wherein R¹ is ethyl and R² is methyl and L is a trimethylstannyl group.

12. A process of claim 6 wherein the radiohalogen is radioiodine (¹²³I, ¹²⁴I, ¹²⁵I ¹³¹I).

13. A process of claim 6 wherein the radiohalogen is ⁷⁶Br or ⁸²Br.

14. A process of claim 6 wherein the radiohalogen is ¹⁸F.
